(19) United States
(12) Patent Application Publication
(10) Pub. No.: US 2026/0370212 A1
(43) Pub. Date: Nov. 19, 2026

(54) SURFACE ACOUSTIC WAVE DEVICE BASED SENSOR PACKAGE

(71) Applicant: WISOL CO., LTD., Osan-si (KR)

(72) Inventors: Jung Min KIM, Osan-si (KR); Yu Rim CHOI, Osan-si (KR)

(73) Assignee: WISOL CO., LTD., Osan-si (KR)

(21) Appl. No.: 19/669,430

(22) Filed: May 6, 2026

(30) Foreign Application Priority Data
May 13, 2025 (KR) 10-2025-0062193

(51) Int. Cl. H03H 9/02; G01N 29/02; H03H 9/05; H03H 3/08

(52) U.S. Cl. CPC H03H 9/02992; G01N 29/022; H03H 9/059; H03H 3/08

(57) ABSTRACT

A sensor package based on a surface acoustic wave device is provided. The sensor package based on a surface acoustic wave device includes a substrate and a sensor disposed on one surface of the substrate, wherein the sensor includes a die including a device arrangement surface connected to the one surface of the substrate by at least one solder, and a plurality of IDT electrodes arranged side by side on the device arrangement surface to face the one surface of the substrate, each of the plurality of IDT electrodes having a sensing layer formed on at least a portion of a surface thereof.

### FIELD OF THE INVENTION

The present invention relates to a sensor package based on a surface acoustic wave device, and more particularly, to a sensor package including a sensor based on a surface acoustic wave device configured to detect a specific substance by converting a state change of an acoustic wave propagating along a surface of the sensor into an electrical signal.

### BACKGROUND OF THE RELATED ART

A surface acoustic wave (SAW) refers to a wave propagating along a surface of an elastic solid, and such a surface acoustic wave is a mechanical wave in which energy is concentrated near the surface during propagation. A surface acoustic wave device is an electromechanical device that utilizes an interaction between the surface acoustic wave and conduction electrons, and uses the surface acoustic wave transmitted on a surface of a piezoelectric crystal. Such a surface acoustic wave device may have a wide range of industrial applications, including a sensor, an oscillator, and a filter, and may have various advantages such as miniaturization and weight reduction, robustness, stability, sensitivity, low cost, and real-time response.

In particular, recently, a sensor based on a surface acoustic wave device capable of detecting a specific substance has been introduced, in which a sensing layer (for example, including a biomolecule such as an antibody or an enzyme) that reacts with the specific substance, such as a virus or a toxic material, is formed on a surface of an IDT (Interdigital Transducer) electrode.

### Technical problem

A technical problem to be solved by the present invention is to provide a sensor package including a sensor based on a surface acoustic wave device configured to detect a specific substance by converting a state change of an acoustic wave propagating along a surface of the sensor into an electrical signal.

The technical problems of the present invention are not limited to the above-mentioned technical problem, and other technical problems not mentioned herein will be clearly understood by those skilled in the art from the following description.

### Technical solution

A sensor package based on a surface acoustic wave device according to an embodiment of the present invention for solving the above technical problem includes a substrate; and a sensor disposed on one surface of the substrate, wherein the sensor includes a die including a device arrangement surface connected to the one surface of the substrate by at least one solder; and a plurality of IDT electrodes arranged side by side on the device arrangement surface to face the one surface of the substrate, each of the plurality of IDT electrodes having a sensing layer formed on at least a portion of a surface thereof.

In some embodiments of the present invention, the sensor may include a test solution injection gap formed between the die and the substrate by the solder.

In some embodiments of the present invention, when a test solution is dropped on the one surface of the substrate, the test solution may move through the test solution injection gap by a capillary action to contact the plurality of IDT electrodes.

In some embodiments of the present invention, the substrate may include a via hole penetrating the substrate and overlapping the sensor in a vertical direction.

In some embodiments of the present invention, the via hole may be hollow, such that when a test solution is dropped on the other surface of the substrate opposite to the one surface, the test solution may move to the test solution injection gap through the via hole by a capillary action.

In some embodiments of the present invention, the sensor may measure a change in frequency caused by a target to be detected included in the test solution being attached to the plurality of IDT electrodes.

A sensor package based on a surface acoustic wave device according to another embodiment of the present invention for solving the above technical problem includes a substrate; and a first sensor and a second sensor disposed on one surface of the substrate, wherein the first sensor includes a first die including a device arrangement surface connected to the one surface of the substrate by at least one first solder; and a plurality of first IDT electrodes arranged side by side on the device arrangement surface to face the one surface of the substrate, each of the plurality of first IDT electrodes having a first sensing layer formed on at least a portion of a surface thereof, and the second sensor includes a second die including a device arrangement surface connected to the one surface of the substrate by at least one second solder; and a plurality of second IDT electrodes arranged side by side on the device arrangement surface to face the one surface of the substrate, each of the plurality of second IDT electrodes having a second sensing layer formed on at least a portion of a surface thereof, the second sensing layer being different in type from the first sensing layer.

In some embodiments of the present invention, the first sensor may include a first test solution injection gap formed between the first die and the substrate by the first solder, and the second sensor may include a second test solution injection gap formed between the second die and the substrate by the second solder.

In some embodiments of the present invention, when a test solution is dropped on a test region defined on the one surface of the substrate, the test solution may move to the first test solution injection gap and the second test solution injection gap, respectively, by a capillary action to contact the plurality of first IDT electrodes and the plurality of second IDT electrodes, respectively.

In some embodiments of the present invention, the test region may be interposed between the first sensor and the second sensor.

In some embodiments of the present invention, the substrate may include a first via hole penetrating the substrate and overlapping the first sensor in a vertical direction, and a second via hole penetrating the substrate and overlapping the second sensor in a vertical direction.

In some embodiments of the present invention, the first via hole and the second via hole may be hollow, such that when a test solution is dropped on the other surface of the substrate opposite to the one surface, the test solution may move through the first via hole and the second via hole, respectively, by a capillary action to move to the first test solution injection gap and the second test solution injection gap, respectively.

In some embodiments of the present invention, the other surface of the substrate may be exposed to the outside so that the test solution can be dropped thereon, and the first die and the second die may be located on a bottom surface.

In some embodiments of the present invention, the first sensor and the second sensor may measure a change in frequency caused by a target to be detected included in the test solution being attached to the plurality of first IDT electrodes and the plurality of second IDT electrodes.

Details of other embodiments are included in the detailed description and the drawings.

### Effects of the invention

The sensor package based on a surface acoustic wave device according to an embodiment of the present invention may detect a target to be detected by measuring a change in frequency caused by the target to be detected included in a test solution being attached on a plurality of IDT electrodes.

In the sensor package of the present invention, as the sensor is connected to the substrate through flip-chip bonding, the IDT electrodes may be less susceptible to durability issues that may occur when the IDT electrodes are exposed to the outside. In this case, in order to deliver the test solution to the IDT electrodes that are not exposed, a method of moving the test solution to a gap formed by solder by a capillary action is used.

In addition, by injecting a coating solution for forming a sensing layer using a via hole formed in the substrate, a possibility of damage to the sensing layer due to a reflow process may be eliminated.

The effects of the present invention are not limited to the above-mentioned effects, and other effects not mentioned herein will be clearly understood by those skilled in the art from the appended claims.

### BRIEF DESCRIPTION OF THE DRAWINGS

FIG. 1 is a view of a sensor package based on a surface acoustic wave device according to some embodiments of the present invention.

FIG. 2 is a cross-sectional view taken along line A-A′ of FIG. 1.

FIG. 3 is a view for explaining a detection process using a test solution in the sensor package based on a surface acoustic wave device of FIGS. 1 and 2.

FIG. 4 is a view of a sensor package based on a surface acoustic wave device according to other embodiments of the present invention.

FIG. 5 is a cross-sectional view taken along line A-A′ of FIG. 4.

FIG. 6 is a view for explaining an injection process of a coating solution and a test solution in the sensor package based on a surface acoustic wave device of FIGS. 4 and 5.

FIG. 7 is a view of a sensor package based on a surface acoustic wave device according to still other embodiments of the present invention.

FIG. 8 is a cross-sectional view taken along line A-A′ of FIG. 7.

FIG. 9 is a view for explaining an injection process of a coating solution and a test solution in the sensor package based on a surface acoustic wave device of FIGS. 7 and 8.

FIG. 10 is a flowchart for explaining a method of manufacturing a sensor package based on a surface acoustic wave device according to an embodiment of the present invention.

### EMBODIMENTS OF THE INVENTION

The advantages and features of the present invention, and the methods of achieving them, will become apparent from the following detailed description of exemplary embodiments with reference to the accompanying drawings. However, the present invention is not limited to the embodiments disclosed herein but may be embodied in various different forms. The embodiments are provided so as to fully convey the scope of the invention to those skilled in the art and to ensure a complete understanding of the disclosure. The scope of the invention is defined only by the claims. Throughout the specification, the same reference numerals refer to the same components.

When an element is referred to as being “connected to” or “coupled to” another element, it can be directly connected or coupled to the other element, or indirectly connected or coupled thereto with one or more intervening elements. In contrast, when an element is referred to as being “directly connected to” or “directly coupled to” another element, there are no intervening elements present. The term “and/or” includes any and all combinations of the associated items.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. The terms “comprises” and/or “comprising,” when used in this specification, specify the presence of stated features, integers, steps, operations, elements, and/or components, but do not preclude the presence or addition of one or more other features, integers, steps, operations, elements, and/or components.

Although terms such as “first,” “second,” and the like may be used to describe various elements, such terms are used merely for convenience of distinction and do not imply any limitation or order. Thus, a “first” element may also be referred to as a “second” element within the technical scope of the invention.

Unless otherwise defined, all terms used herein (including technical and scientific terms) have the same meaning as commonly understood by one of ordinary skill in the art to which the invention pertains. Terms defined in commonly used dictionaries are to be interpreted as having meanings consistent with their contextual use in the relevant art and are not to be interpreted in an idealized or overly formal sense unless expressly so defined herein.

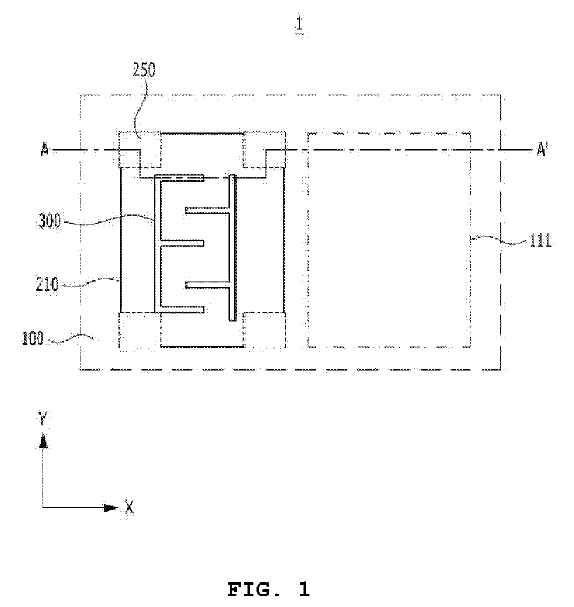

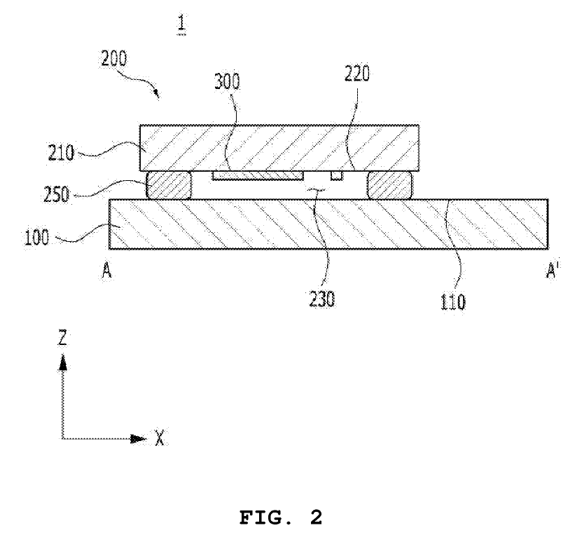

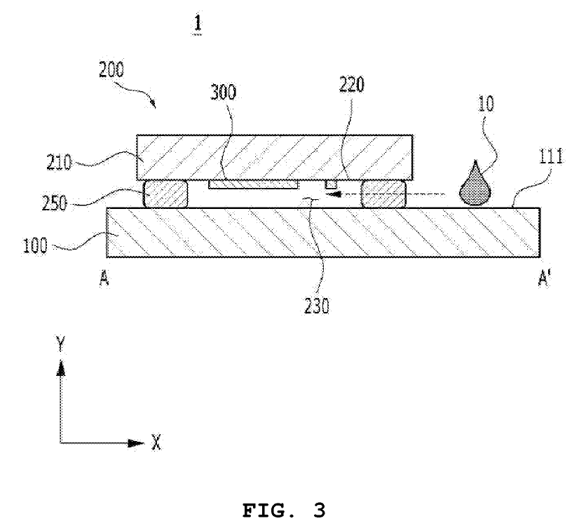

FIG. 1 is a view of a sensor package based on a surface acoustic wave device according to some embodiments of the present invention, FIG. 2 is a cross-sectional view taken along line A-A′ of FIG. 1, and FIG. 3 is a view for explaining a detection process using a test solution in the sensor package based on a surface acoustic wave device of FIGS. 1 and 2.

Referring to FIGS. 1 and 2, a sensor package 1 based on a surface acoustic wave device according to an embodiment of the present invention may include a substrate 100 and a first sensor 200.

The substrate 100 supports the first sensor 200, and may include a lead frame, a PCB (Printed Circuit Board), or the like. Meanwhile, although not shown in FIG. 1, a peripheral circuit and/or a semiconductor chip electrically connected to the first sensor 200 may be additionally disposed on the substrate 100.

A mounting region of the first sensor 200 may be defined on one surface 110 of the substrate 100. That is, the mounting region is a region overlapping the first sensor 200 and the one surface 110 of the substrate in a third direction Z.

The first sensor 200 may include a biosensor based on a surface acoustic wave. That is, the first sensor 200 may include a plurality of IDT electrodes 300 disposed on a device arrangement surface 220 and facing the one surface 110 of the substrate, and may be a sensor configured to detect a target to be detected by measuring a change in frequency caused by the target to be detected included in a test solution being attached to a sensing layer on the plurality of IDT electrodes 300. Here, the test solution may include a body fluid collected from a human body, and the target to be detected may include an antigen such as a virus, a drug, or a pathogen, or an antibody generated by a body in response thereto.

The plurality of IDT electrodes 300 may include an input-side IDT electrode and an output-side IDT electrode extending in a first direction X on a piezoelectric substrate and spaced apart from each other in a second direction Y. In some embodiments of the present invention, a sensing layer may be formed on at least a portion of a surface of the plurality of IDT electrodes 300. Here, the sensing layer may include, for example, an antigen (when a target to be detected includes an antibody) or an antibody (when a target to be detected includes an antigen) to induce an antigen–antibody binding. In some embodiments, the antigen or the antibody included in the sensing layer may be immobilized on the surface of the plurality of IDT electrodes 300 by BSA (Bovine Serum Albumin) or the like.

The surface of the plurality of IDT electrodes 300 may include a metal that facilitates formation of the sensing layer, for example, gold (Au).

In FIG. 1 and the like, the number of the plurality of IDT electrodes 300 included in the first sensor 200 is schematically illustrated for convenience of description, and an actual number of the plurality of IDT electrodes 300 may be several tens to several hundreds.

The plurality of IDT electrodes 300 may be disposed on a die 210. The die 210 may include a piezoelectric substrate such as LiTaO3 (LT), LiNbO3 (LN), or single-crystal quartz that propagates a surface acoustic wave generated by the plurality of IDT electrodes 300. In addition, the die 210 may include at least one stacked structure of a high-velocity layer having a propagation velocity higher than that of the surface acoustic wave propagating through the piezoelectric substrate, a low-velocity layer having a lower propagation velocity, and a supporting substrate supporting the same.

The first sensor 200 may be bonded on the one surface 110 of the substrate 100 by a solder 250 in a flip-chip bonding method. Accordingly, external exposure of the plurality of IDT electrodes 300 disposed on the die 210 may be minimized.

The solder 250 may mechanically and electrically connect the first sensor 200 and the substrate 100.

The solder 250 formed through an SMT (Surface Mount Technology) process may have a thickness of several tens to several hundreds of micrometers, and thus a space having a predetermined gap may be formed between the first sensor 200 and the substrate 100. In the present specification, such a space between the first sensor 200 and the substrate 100 is defined as a test solution injection gap 230.

The test solution injection gap 230 may have a gap of several tens to several hundreds of micrometers, similar to the thickness of the solder 250, and this gap may correspond to a vertical distance between the device arrangement surface 220 of the die 210 and the one surface 110 of the substrate 100.

The test solution injection gap 230 and a test method using the same will be described with additional reference to FIG. 3.

FIG. 3 is a view for explaining a test solution injection and detection method in a sensor package based on a surface acoustic wave device according to some embodiments of the present invention.

Referring to FIG. 3, in the sensor package 1 based on a surface acoustic wave device according to an embodiment of the present invention, the test solution injection gap 230 may serve as a path through which a test solution 10 to be detected moves.

Specifically, the test solution injection gap 230 may serve as a path along which the test solution 10 dropped on one surface 110 of the substrate 100, particularly on a test region 111 defined adjacent to the sensor 200, moves by a capillary action to contact the plurality of IDT electrodes 300 on the device arrangement surface 220. That is, the thickness of the test solution injection gap 230 is formed to be several tens to several hundreds of micrometers, which is sufficiently small to induce a capillary action, such that the test solution 10 may be drawn into the test solution injection gap 230 and contact the plurality of IDT electrodes 300 without a separate means for inducing movement of the test solution 10.

Accordingly, in order to allow movement of the test solution 10 as described above, the test solution injection gap 230 may remain as an empty space without a separate underfill.

If an antigen or an antibody related to a target to be detected is included in the test solution 10, the antigen or the antibody may bind to an antibody or an antigen of a sensing layer formed on a surface of the plurality of IDT electrodes 300, and this may appear as a change in frequency and be detected by the first sensor 200.

In the sensor package 1 based on a surface acoustic wave device according to an embodiment of the present invention, as the first sensor 200 is mounted on the substrate 100 by flip-chip bonding, exposure of the plurality of IDT electrodes 300 coated with a sensing layer sensitive to external environmental conditions may be minimized. In addition, by using a capillary action of the test solution injection gap 230 formed by the solder 250 in order to provide the test solution 10 to the plurality of IDT electrodes 300 in an environment separated from the outside, the test solution 10 and the plurality of IDT electrodes 300 may be brought into contact without a separate means for providing the test solution 10.

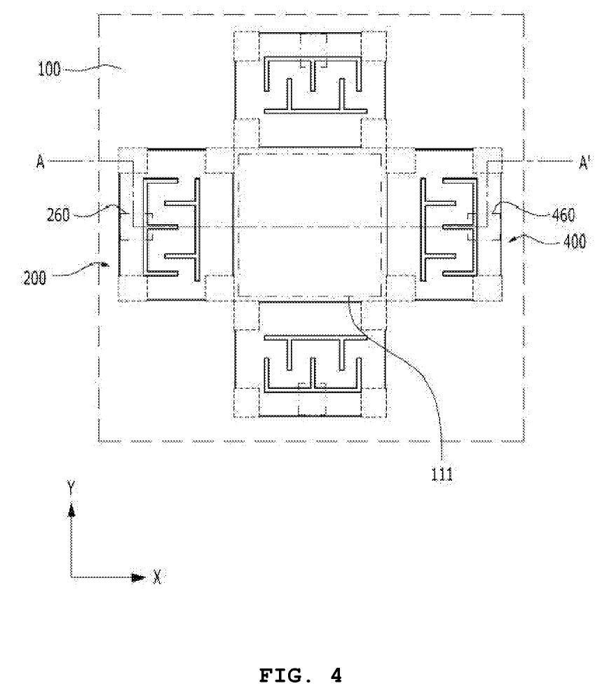

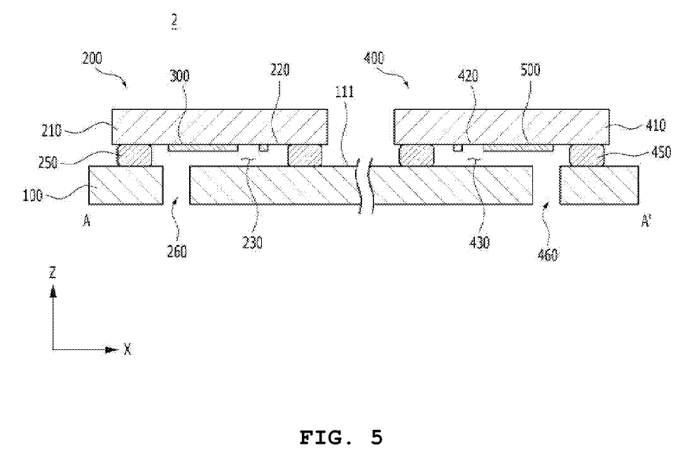

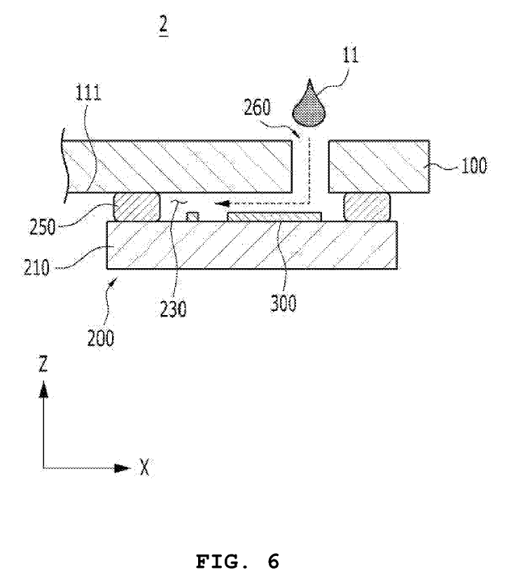

FIG. 4 is a view of a sensor package based on a surface acoustic wave device according to other embodiments of the present invention, FIG. 5 is a cross-sectional view taken along line A-A′ of FIG. 4, and FIG. 6 is a view for explaining an injection process of a coating solution and a test solution in the sensor package based on a surface acoustic wave device of FIGS. 4 and 5.

Referring to FIGS. 4 and 5, a sensor package 2 based on a surface acoustic wave device according to an embodiment of the present invention is characterized in that the substrate 100 includes a hole through which the substrate 100 communicates with the outside, that is, via holes 260 and 460.

Specifically, a via hole 260 may be formed in the substrate 100 at a position overlapping the sensor 200 in a third direction Z. The via hole 260 may preferably overlap the plurality of IDT electrodes 300 in the third direction Z and may be formed at a position that does not overlap the solder 250.

In order to form a sensing layer on the surface of the plurality of IDT electrodes 300 in the sensor package 1 and 2 based on a surface acoustic wave device of the present invention, a coating solution including an antigen or an antibody needs to reach the surface of the plurality of IDT electrodes 300, and after a predetermined time elapses, the antigen or the antibody needs to be attached to the surface of the plurality of IDT electrodes 300.

However, as described above, when the sensor 200 is connected to the substrate 100 by an SMT process, a reflow process for fixing the solder 250 needs to be performed. Since the reflow process is performed under a temperature condition ranging from 200 degrees to 250 degrees, a sensing layer including various proteins may be damaged during the SMT process.

Accordingly, the sensing layer formed on the surface of the plurality of IDT electrodes 300 needs to be formed after the sensor 200 and the substrate 100 are connected, and thus the coating solution is injected through the via hole 260 penetrating the substrate 100 to allow the coating solution to reach the surface of the plurality of IDT electrodes 300.

The via hole 260 may have a diameter of several tens to several hundreds of micrometers, may penetrate the substrate 100, and may be hollow. Accordingly, a space between the substrate 100 and the sensor 200, that is, the test solution injection gap 230, may be connected to the outside of the package 2 through the via hole 260.

Referring to FIG. 6, a schematic view illustrating a process in which a coating solution 11 is dropped into the via hole 260 and reaches the surface of the plurality of IDT electrodes 300 is shown. For ease of understanding, FIG. 6 is illustrated by inverting a portion of FIG. 5.

When the coating solution 11 is dropped at a position of the via hole 260 in the substrate 100, the coating solution 11 proceeds along the via hole 260 to the test solution injection gap 230 by a capillary action caused by the via hole 260, and spreads within the test solution injection gap 230 to contact the plurality of IDT electrodes 300. Thereafter, when the coating solution 11 is dried, a sensing layer including an antigen or an antibody may be formed on the surface of the plurality of IDT electrodes 300.

The test solution may be dropped on the test region 111 as in FIG. 3, and then proceed along the test solution injection gap 230. The test solution may react with the sensing layer on the surface of the plurality of IDT electrodes 300 within the test solution injection gap 230, and when an antigen–antibody binding occurs by being attached to the sensing layer, the sensor 200 may detect whether a frequency of a surface acoustic wave changes.

Meanwhile, the sensor package 2 based on a surface acoustic wave device according to an embodiment of the present invention may include a plurality of sensors 200 and 400 connected to the substrate 100.

The first sensor 200 and the second sensor 400 may be disposed on the substrate 100 to be spaced apart from each other with a test region 111 interposed therebetween. The second sensor 400 may include a plurality of IDT electrodes 500 disposed on a device arrangement surface 420 of a die 410, and a test solution injection gap 430 may be formed as the second sensor 400 is connected to the substrate 100 by a solder 450.

In FIG. 4, a total of four sensors, including the first sensor 200 and the second sensor 400, are illustrated as being disposed on the substrate 100 around the test region 111. However, the present invention is not limited thereto, and the number of sensors disposed on the substrate 100 may vary depending on a target to be detected, for example, a type of virus, bacteria, or drug to be detected by a single injection of the test solution. When four sensors are disposed around the test region 111 as shown in FIG. 4, the sensor package may correspond to a sensor package configured to detect a total of four targets to be detected by a single injection of the test solution.

An arrangement structure of the sensors disposed around the test region 111 may vary depending on the number of sensors. When n sensors are disposed around the test region 111, a virtual n-sided polygon may be formed around the test region 111, and one sensor may be disposed on each side of the n-sided polygon. When only two sensors are disposed, the sensors may be disposed side by side with the test region 111 interposed therebetween.

When four sensors are disposed as shown in FIG. 4, the test region 111 may have, for example, a rectangular or square shape having a width and a length of several millimeters. Of course, the width and the length of the test region 111 may vary depending on sizes of the sensors 200 and 400. In the test region 111, a surface of the substrate 100 on which no separate circuit element is disposed may be exposed.

When the test solution is dropped on the test region 111, the test solution may proceed along the test solution injection gaps 230 and 430 included in the first sensor 200 and the second sensor 400 disposed around the test region 111, respectively.

Different types of sensing layers may be formed on the surfaces of the plurality of first IDT electrodes 300 and the plurality of second IDT electrodes 500. Here, the different types of sensing layers mean sensing layers to which different antigens or antibodies are attached in order to detect different targets to be detected. Accordingly, in a manufacturing process of the sensor package 2, the respective sensing layers may be formed by injecting different coating solutions into the respective via holes 260 and 460.

As described above, in a test process for detecting a target to be detected, detection for different sensors 200 and 400 may be simultaneously performed by a single drop of the test solution onto the test region 111.

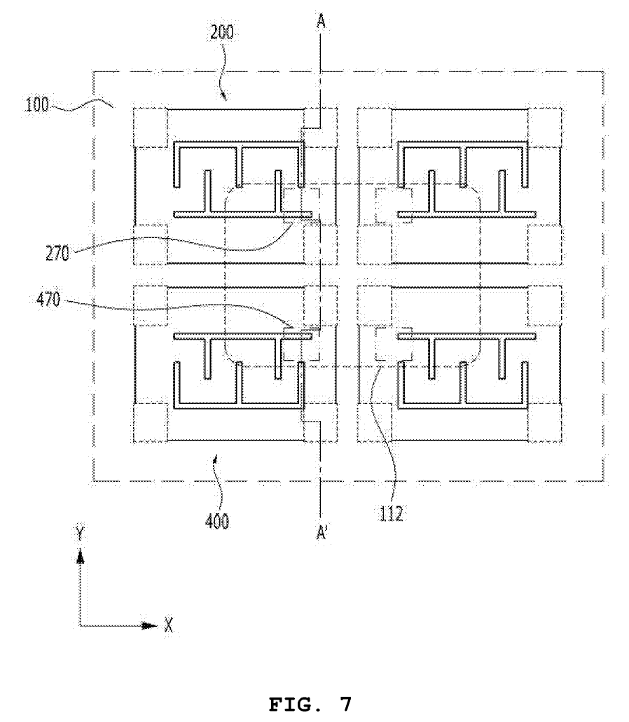

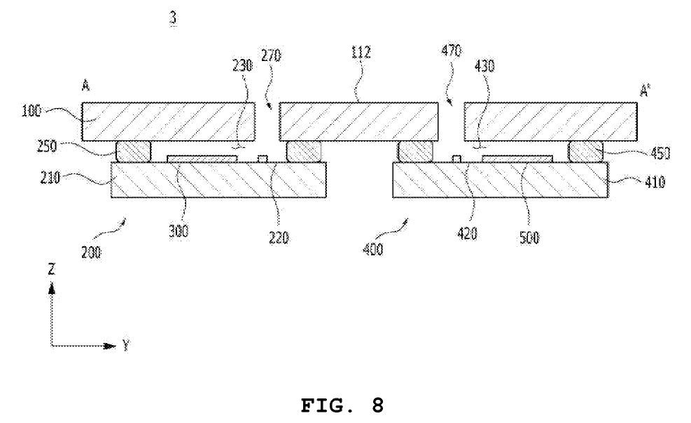

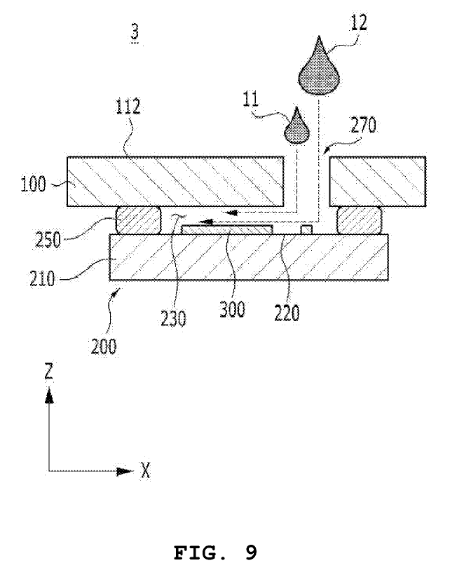

FIG. 7 is a view of a sensor package based on a surface acoustic wave device according to still other embodiments of the present invention, FIG. 8 is a cross-sectional view taken along line A-A′ of FIG. 7, and FIG. 9 is a view for explaining an injection process of a coating solution and a test solution in the sensor package based on a surface acoustic wave device of FIGS. 7 and 8.

Referring to FIGS. 7 and 8, a sensor package 3 based on a surface acoustic wave device according to still other embodiments of the present invention may have an arrangement structure of sensors different from that of the sensor package 2 described above with reference to FIGS. 4 to 6, and an injection method of a coating solution and a test solution may also be different.

In particular, in the sensor package 3 based on a surface acoustic wave device according to the present embodiment, the substrate 100 may be oriented such that another surface not facing the sensor 200 is exposed to the outside, and a test may be performed by dropping a test solution on a test region 112 defined on the other surface. That is, when the product is mounted in an enclosure or the like, the substrate 100 is positioned upward and the sensors 200 and 400 are in contact with a bottom surface of the enclosure, such that the dies 210 and 410 of the sensors are not exposed to the outside, thereby improving product reliability.

As illustrated, the test region 111 described above is not interposed between the first sensor 200 and the second sensor 400. Accordingly, the first sensor 200 and the second sensor 400 may be spaced apart from each other with only a small gap for separating circuit elements.

Via holes 270 and 470 corresponding to the respective sensors 200 and 400 may be formed in the substrate 100. The via holes 270 and 470 may penetrate the substrate 100 and may be formed at positions overlapping the sensors 200 and 400, preferably the plurality of IDT electrodes 300 and 500, in the third direction Z and not overlapping the solders 250 and 450, as in the above-described embodiments.

Referring to FIG. 9, in the sensor package 3 based on a surface acoustic wave device according to the present embodiment, both a coating solution 11 and a test solution 12 may move through the via hole 270. That is, in a manufacturing process of the sensor package 3, the coating solution 11 is injected through the via hole 270, and the coating solution 11 moves along the test solution injection gap 230 to reach the surface of the plurality of IDT electrodes 300. At this time, different types of sensing layers may be formed for different sensors 200 and 400 by injecting different coating solutions through the via holes 270 and 470, as in the above-described embodiments.

In a detection process for a target to be detected, the test solution 12 may be dropped within the test region 112 and may move along the via hole 270 and the test solution injection gap 230. As illustrated, when four different sensors are disposed, four sensors may simultaneously perform detection for different targets by a single drop of the test solution.

The sensor package 3 may include n sensors, and an arrangement structure of the n sensors may be arranged in a maximally dense structure according to sizes of the sensors without considering a test region on which the test solution is dropped, unlike the above-described embodiments.

The via holes 270 and 470 corresponding to the respective sensors may also be formed to overlap the sensors in the third direction Z, but may be formed to be gathered at positions corresponding to the test region 112 on the other surface of the substrate.

Meanwhile, in another embodiment of the present invention, unlike FIG. 7, only a single sensor (for example, 200) may be formed on the substrate 100. In this case, the sensor 200 and the die 210 are located below, and injection of the coating solution for forming a sensing layer and injection of the test solution may be performed through the via hole 270 formed in the substrate 100.

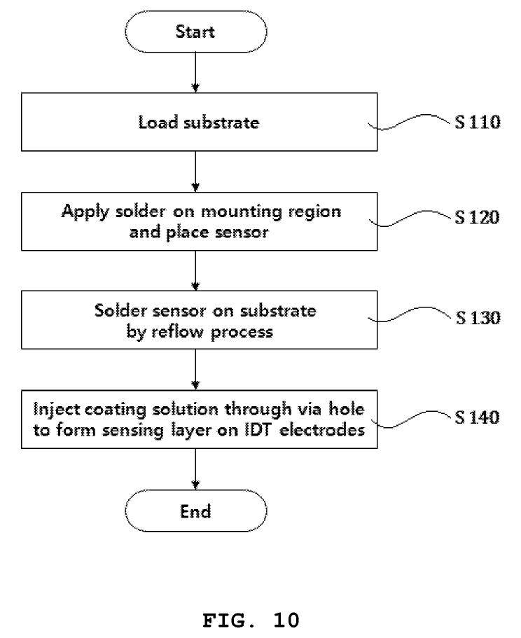

FIG. 10 is a flowchart for explaining a method of manufacturing a sensor package based on a surface acoustic wave device according to an embodiment of the present invention.

Referring to FIG. 10, the method of manufacturing the sensor package based on a surface acoustic wave device according to an embodiment of the present invention may include loading a substrate (S110), applying a solder on a mounting region of the substrate and placing a sensor (S120), soldering the sensor on the substrate by a reflow process (S130), and injecting a coating solution through a via hole of the substrate to form a sensing layer on an IDT electrode (S140).

From the loading of the substrate to the soldering step (S110 to S130), the process may be performed according to a generally known SMT process.

In step S140, forming a sensing layer on at least a portion of a plurality of IDT electrodes may be performed. As illustrated in FIGS. 6 and 9, a coating solution for forming a sensing layer may be injected through a via hole 260 and 270 at a position overlapping the sensors 200 and 400 of the substrate 100. Through this method, durability may be improved by preventing external exposure of the plurality of IDT electrodes 300 of the flip-chip bonded sensor 200, and damage to the sensing layer due to heat may be prevented by forming the sensing layer after the reflow process.

Although embodiments of the present invention have been described above with reference to the accompanying drawings, those skilled in the art will understand that the present invention may be implemented in other specific forms without changing the technical spirit or essential features thereof. Therefore, the embodiments described above should be understood to be illustrative in all respects and not limiting.

1. A sensor package based on a surface acoustic wave device, comprising:
a substrate; and
a sensor disposed on one surface of the substrate,
wherein the sensor includes
a die including a device arrangement surface connected to the one surface of the substrate by at least one solder; and
a plurality of IDT electrodes arranged side by side on the device arrangement surface to face the one surface of the substrate, each of the plurality of IDT electrodes having a sensing layer formed on at least a portion of a surface thereof.

2. The sensor package based on a surface acoustic wave device of claim 1, wherein the sensor includes a test solution injection gap formed between the die and the substrate by the solder.

3. The sensor package based on a surface acoustic wave device of claim 2, wherein, when a test solution is dropped on the one surface of the substrate, the test solution moves through the test solution injection gap by a capillary action to contact the plurality of IDT electrodes.

4. The sensor package based on a surface acoustic wave device of claim 2, wherein the substrate includes a via hole penetrating the substrate and overlapping the sensor in a vertical direction.

5. The sensor package based on a surface acoustic wave device of claim 4, wherein the via hole is hollow, such that, when a test solution is dropped on another surface of the substrate opposite to the one surface, the test solution moves through the via hole by a capillary action to the test solution injection gap.

6. The sensor package based on a surface acoustic wave device of claim 1, wherein the sensor measures a change in frequency caused by a target to be detected included in a test solution being attached to the plurality of IDT electrodes.

7. A sensor package based on a surface acoustic wave device, comprising: a substrate; and
a first sensor and a second sensor disposed on one surface of the substrate,
wherein the first sensor includes
a first die including a device arrangement surface connected to the one surface of the substrate by at least one first solder; and
a plurality of first IDT electrodes arranged side by side on the device arrangement surface to face the one surface of the substrate, each of the plurality of first IDT electrodes having a first sensing layer formed on at least a portion of a surface thereof, and
the second sensor includes
a second die including a device arrangement surface connected to the one surface of the substrate by at least one second solder; and
a plurality of second IDT electrodes arranged side by side on the device arrangement surface to face the one surface of the substrate, each of the plurality of second IDT electrodes having a second sensing layer formed on at least a portion of a surface thereof, the second sensing layer being different in type from the first sensing layer.

8. The sensor package based on a surface acoustic wave device of claim 7, wherein the first sensor includes a first test solution injection gap formed between the first die and the substrate by the first solder, and
the second sensor includes a second test solution injection gap formed between the second die and the substrate by the second solder.

9. The sensor package based on a surface acoustic wave device of claim 8, wherein, when a test solution is dropped on a test region defined on the one surface of the substrate, the test solution moves to the first test solution injection gap and the second test solution injection gap, respectively, by a capillary action to contact the plurality of first IDT electrodes and the plurality of second IDT electrodes, respectively.

10. The sensor package based on a surface acoustic wave device of claim 9, wherein the test region is interposed between the first sensor and the second sensor.

11. The sensor package based on a surface acoustic wave device of claim 8, wherein the substrate includes a first via hole penetrating the substrate and overlapping the first sensor in a vertical direction, and a second via hole penetrating the substrate and overlapping the second sensor in a vertical direction.

12. The sensor package based on a surface acoustic wave device of claim 11, wherein the first via hole and the second via hole are hollow, such that, when a test solution is dropped on another surface of the substrate opposite to the one surface, the test solution moves through the first via hole and the second via hole, respectively, by a capillary action to the first test solution injection gap and the second test solution injection gap, respectively.

13. The sensor package based on a surface acoustic wave device of claim 12, wherein the other surface of the substrate is exposed to the outside so that the test solution is dropped thereon, and the first die and the second die are located on a bottom surface.

14. The sensor package based on a surface acoustic wave device of claim 7, wherein the first sensor and the second sensor measure a change in frequency caused by a target to be detected included in a test solution being attached to the plurality of first IDT electrodes and the plurality of second IDT electrodes.
